(19) United States
(12) Patent Application Publication
(10) Pub. No.: US 2021/0100523 A1
(43) Pub. Date: Apr. 8, 2021

(54) DETERMINATION OF BLOOD VESSEL CHARACTERISTIC CHANGE USING AN ULTRASONIC SENSOR

(71) Applicant: InvenSense, Inc., San Jose, CA (US)

(72) Inventors: Xiaoyue JIANG, San Jose, CA (US); Peter George HARTWELL, Menlo Park, CA (US)

(73) Assignee: InvenSense, Inc., San Jose, CA (US)

(21) Appl. No.: 17/062,404

(22) Filed: Oct. 2, 2020

Related U.S. Application Data
(60) Provisional application No. 62/911,083, filed on Oct. 4, 2019.

(51) Int. Cl. A61B 8/06; A61B 8/08; A61B 8/04

(52) U.S. Cl. CPC A61B 8/06; A61B 8/0891; A61B 8/04; A61B 8/488

(57) ABSTRACT

In a method for determining blood vessel characteristic change using an ultrasonic sensor, a plurality of ultrasonic signal transmit and receive operations are performed at a position overlying a blood vessel of a person using an ultrasonic sensor, where the plurality of ultrasonic signal transmit and receive operations generate a plurality of received signals. Depths of blood vessel walls are determined at the position for a plurality of time instances based on local maxima of a combination of an acoustic impedance mismatch and a motion characteristic based at least in part on the plurality of received signals. A change in a blood vessel characteristic is determined based at least in part on a difference between the depths of the blood vessel walls at the plurality of time instances.

### RELATED APPLICATION

This application claims also priority to and the benefit of co-pending U.S. Provisional Patent Application 62/911,083, filed on Oct. 4, 2019, entitled “ULTRASONIC WELLNESS SENSOR,” by Xiaoyue Jiang, having Attorney Docket No. IVS-934-PR, and assigned to the assignee of the present application, which is incorporated herein by reference in its entirety.

### BACKGROUND

The development of consumer electronics has enabled the possibility to address the need of people's increasing awareness of their health and wellness. For example, wearable devices and smart phones have been able to host various sensor modalities for cardiovascular system monitoring, e.g., integrated electrodes for electrocardiogram (ECG), optical sensors for photoplethysmography (PPG), and pressure sensors for blood pressure. This enables people to measure parameters that can be used as an indicator for wellness themselves, for example at home without the need of a medical professional, or in the form of in-home care with the help of a medical professional. However, the ability of people to monitor their health and wellness, such as to monitor parameters of the cardiovascular system like electrical potential, pressure, or blood flow, depends on the available sensors, their ease of use, and their accuracy. Moreover, often the measurements are reflecting an averaged information over time or over (parts of) the body, lacking the details and/or fluctuations that may be useful for the monitoring process.

### BRIEF DESCRIPTION OF THE DRAWINGS

The accompanying drawings, which are incorporated in and form a part of the Description of Embodiments, illustrate various non-limiting and non-exhaustive embodiments of the subject matter and, together with the Description of Embodiments, serve to explain principles of the subject matter discussed below. Unless specifically noted, the drawings referred to in this Brief Description of Drawings should be understood as not being drawn to scale and like reference numerals refer to like parts throughout the various figures unless otherwise specified.

FIG. 1 illustrates a representation of a pressure wave moving through a blood vessel and blood vessel wall velocity caused by blood flow in a flexible blood vessel, according to some embodiments.

FIG. 2 illustrates an example graph measurement of tissue velocity for use in determining blood vessel wall depth, according to some embodiments.

FIG. 3 illustrates example graphs of variations in relative position of blood vessel walls of a blood vessel over time, a change in diameter of the blood vessel over time, and heart beats per minute, according to some embodiments.

FIG. 4A illustrates a block diagram of an example system for determining blood vessel characteristic change using an ultrasonic sensor, according to some embodiments.

FIG. 4B illustrates a block diagram of an example system for determining blood vessel wall depth, according to some embodiments.

FIG. 4C illustrates a block diagram of an example system for determining blood pressure, according to some embodiments.

FIG. 5 illustrates an example fast time and slow time graphs of received signals for use in determining blood vessel wall depth using ultrasonic sensing system positioned over tissue and a blood vessel, according to some embodiments.

FIG. 6 illustrates example fast time and slow time graphs of signal strength, tissue velocity, and weight tissue velocity for use in determining blood vessel wall depth, according to some embodiments.

FIG. 7 illustrates example graphs for identifying local maxima of a combination of an acoustic impedance mismatch and a motion characteristic based the received ultrasonic signals for identifying the depth of the blood vessel walls relative to the ultrasonic sensor, according to some embodiments.

FIG. 8 illustrates example graphs of relative displacement of the blood vessel walls and the change in diameter of the blood vessel over time, according to some embodiments.

FIGS. 9A, 9B, and 9C illustrate different embodiments of a wellness sensing system for extracting pulse wave velocity information.

FIG. 10 illustrates an example layout of ultrasonic transducers of an ultrasonic sensor, according to an embodiment.

FIGS. 11A, 11B, and 11C illustrate different examples of wellness sensors having an ultrasonic sensor for determining blood vessel characteristic change; according to some embodiments.

FIG. 12 is a block diagram of an example electronic device upon which embodiments described herein may be implemented.

FIG. 13 illustrates an example process for determining blood vessel characteristic change using an ultrasonic sensor, according to some embodiments.

FIG. 14 illustrates an example process for determining blood vessel wall depth, according to some embodiments.

### DESCRIPTION OF EMBODIMENTS

The following Description of Embodiments is merely provided by way of example and not of limitation. Furthermore, there is no intention to be bound by any expressed or implied theory presented in the preceding background or in the following Description of Embodiments.

Reference will now be made in detail to various embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

### Notation and Nomenclature

Some portions of the detailed descriptions which follow are presented in terms of procedures, logic blocks, processing and other symbolic representations of operations on data within an electrical device. These descriptions and representations are the means used by those skilled in the data processing arts to most effectively convey the substance of their work to others skilled in the art. In the present application, a procedure, logic block, process, or the like, is conceived to be one or more self-consistent procedures or instructions leading to a desired result. The procedures are those requiring physical manipulations of physical quantities. Usually, although not necessarily, these quantities take the form of acoustic (e.g., ultrasonic) signals capable of being transmitted and received by an electronic device and/or electrical or magnetic signals capable of being stored, transferred, combined, compared, and otherwise manipulated in an electrical device.

It should be borne in mind, however, that all of these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “performing,” “determining,” “detecting,” “integrating,” “calculating,” “correcting,” “providing,” “receiving,” “analyzing,” “confirming,” “displaying,” “presenting,” “using,” “completing,” “instructing,” “comparing,” “executing,” or the like, refer to the actions and processes of an electronic device such as an electrical device.

Embodiments described herein may be discussed in the general context of processor-executable instructions residing on some form of non-transitory processor-readable medium, such as program modules, executed by one or more computers or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, logic, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example ultrasonic sensing system and/or mobile electronic device described herein may include components other than those shown, including well-known components.

Various techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

Various embodiments described herein may be executed by one or more processors, such as one or more motion processing units (MPUs), sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), a programmable logic controller (PLC), a complex programmable logic device (CPLD), a discrete gate or transistor logic, discrete hardware components, or any combination thereof designed to perform the functions described herein, or other equivalent integrated or discrete logic circuitry. The term “processor,” as used herein may refer to any of the foregoing structures or any other structure suitable for implementation of the techniques described herein. As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Moreover, processors can exploit nano-scale architectures such as, but not limited to, molecular and quantum-dot based transistors, switches and gates, in order to optimize space usage or enhance performance of user equipment. A processor may also be implemented as a combination of computing processing units.

In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a combination of an SPU/MPU and a microprocessor, a plurality of microprocessors, one or more microprocessors in conjunction with an SPU core, MPU core, or any other such configuration.

### Overview of Discussion

Discussion begins with a description of an example system for determining blood vessel characteristic change using an ultrasonic sensor. Example operations of a system for determining blood vessel characteristic change using an ultrasonic sensor are then described.

Medical ultrasound technology is currently employed by medical professional for imaging of the vascular system. Based on the images, medical professionals, such as ultrasound technicians, can deduce various forms of information regarding vascular health, such as vascular wall motion tracking, blood flow, or elastic properties of the soft tissues (e.g., elastography). Typical ultrasound systems currently in use are for clinical usage and meant to be operated by specially educated medical experts. Conventional medical ultrasonic systems typically include ultrasound probes with various shapes and form factors for different body parts, and often output images that are then analyzed further. In a clinical setting, the ultrasound system is used by a physician or clinician to align the probes to the physiological sites of interest and diagnose based on the static ultrasound imaging, Doppler imaging, and elastography. Due to the complexity of the biological system and usage of the ultrasound systems, extensive ultrasound imaging and medical training is needed for ultrasound examination and diagnosis.

Technology development over the last decades has resulted in miniaturized ultrasonic transducers as well as ever-increasing data processing power and storage. An example of the currently available miniaturized ultrasonic transducers is the application of ultrasonic fingerprint sensors in mobile devices. Embodiments describe herein provide a miniaturized ultrasonic sensor system for cardiovascular system monitoring. The described system provides a user friendly system that does not necessarily require operation by a trained medical professional, but due to system optimization and signal processing, allows for home usage. For example, the described system can be used by people at home (without medical training), by in-home care personnel, or even by automated home robots or similar autonomous devices. The system can measure and output various parameters of the blood vessels, e.g., blood vessel diameter and time variations, pulse wave velocity, blood pressure, etc. The described system may also output a wellness indicator based on these parameters, and this wellness indicator may be monitored over time.

Embodiments described herein provide a method for determining blood vessel characteristic change using an ultrasonic sensor. A plurality of ultrasonic signal transmit and receive operations is performed at a position overlying a blood vessel of a person using an ultrasonic sensor, wherein the plurality of ultrasonic signal transmit and receive operations generate a plurality of received signals. Depths of blood vessel walls of one blood vessel (e.g., a closer blood vessel wall and a farther blood vessel wall relative to the ultrasonic sensor) are automatically determined at the position for a plurality of time instances based on local maxima of a combination of an acoustic impedance mismatch and a motion characteristic based at least in part on the plurality of received signals. A change in a blood vessel characteristic is determined based at least in part on a difference between the depths of the blood vessel walls at the plurality of time instances. In one embodiment, the blood vessel characteristic is a diameter change of the blood vessel. In another embodiment, the blood vessel characteristic is a blood pressure. In another embodiment, the blood vessel characteristic is a pulse wave velocity of the blood vessel.

In some embodiments, where the motion characteristic is a velocity of tissue, determination of the depths of blood vessel walls includes determining the velocity of the tissue using at least a phase of the received signals. In one embodiment, determination of the velocity of the tissue at the plurality of time instances includes performing Doppler signal processing on the plurality of received signals to determine the velocity of the tissue at the plurality of time instances.

In some embodiments, determination of the depths of blood vessel walls at the position for a plurality of time instances includes determining a weighted velocity of the tissue at the plurality of time instances based on signal amplitudes (e.g., due to an acoustic impedance mismatch) of the plurality of received signals at the plurality of time instances and the velocity of the tissue at the plurality of time instances. In one embodiment, the weighted velocity of the tissue depends on an impact of the acoustic impedance mismatch and on the velocity of the tissue.

In some embodiments, determination of the depths of blood vessel walls at the position for a plurality of time instances based at least in part on the plurality of received signals includes detecting at least one local maximum of the combination of the acoustic impedance mismatch and the motion characteristic based at least in part on the plurality of received signals, wherein the at least one local maximum corresponds to one blood vessel wall. In some embodiment, two local maxima are detected, where one local maximum corresponds to a closer wall of the blood vessel relative to the ultrasonic sensor and the other local maximum corresponds to a farther wall of the blood vessel relative to the ultrasonic sensor. In another embodiment, determination of the depths of blood vessel walls at the position for a plurality of time instances based at least in part on the plurality of received signals includes determining two depth ranges for the blood vessel based on blood vessel geometry, where a first depth range comprises a closer wall of the blood vessel relative to the ultrasonic sensor and a second depth range comprises a farther wall of the blood vessel relative to the ultrasonic sensor. A first local maximum weighted velocity within the first depth range is determined, wherein the first local maximum weighted velocity within the first depth range corresponds to the depth of the closer wall of the blood vessel, and a second local maximum weighted velocity within the second depth range is determined, wherein the second local maximum weighted velocity within the second depth range corresponds to the depth of the farther wall of the blood vessel. In one embodiment, the blood vessel characteristic is a change in blood vessel diameter.

In some embodiments, determination of a change in a blood vessel characteristic based at least in part on a difference between the depths of the blood vessel walls at the plurality of time instances includes determining a velocity of the blood vessel at the depth of the closer wall and a velocity of the blood vessel at the depth of the farther wall at the plurality of time instances, where the velocity of the blood vessel at the depth of the closer wall and the velocity of the blood vessel at the depth of the farther wall are out of phase. The velocity of the blood vessel at the depth of the closer wall and the velocity of the blood vessel at the depth of the farther wall being out of phase can be used as validation that a blood vessel has correctly been found. The velocity of the blood vessel at the depth of the closer wall and the velocity of the blood vessel at the depth of the farther wall is integrated to generate a displacement of the closer wall and a displacement of the farther wall. A change in diameter of the blood vessel is calculated at the plurality of time instances based on a difference of the displacement of the closer wall and the displacement of the farther wall. In addition, by using the time-of-flight (TOF) between the vessel walls to determine the absolute diameter of the blood vessel, the variation in the absolute blood vessel diameter can be determined over time. Moreover, if an additional pressure sensor can obtain pressure at the same location, vascular distensibility and compliance can be derived from pressure and volume change.

In some embodiments, the motion characteristic at tissue at a depth between the ultrasonic sensor and a closer blood vessel wall relative to the ultrasonic sensor is determined. Motion artifacts within displacement of the blood vessel walls are corrected for by using the motion characteristic at tissue at a depth between the ultrasonic sensor and a closer blood vessel wall relative to the ultrasonic sensor.

### Example System for Determining Blood Vessel Characteristic Change Using an Ultrasonic Sensor

As a heart pumps the blood through the vascular system, a pressure wave runs along the blood vessels, which themselves are elastic and flexible. This pressure wave causes the elastic vessels to expand and contract as these pressure waves pass. As a result, there is an expansion wave running along the blood vessels with each heartbeat, where this pressure wave is referred to as the pulse. The pulse wave velocity (PWV) is the velocity at which the blood pressure wave propagates through the circulatory system. As is known by persons skilled in the art, the blood pressure can be calculated from diameter change with some calibration or assumption of the blood vessel geometry (e.g. shape and thickness of the vessel walls) and material properties (e.g. arterial stiffness). The arterial stiffness depends on the pressure, but may be considered a constant as a first order approximation. Under this assumption, there is a linear relationship between the blood pressure and the diameter change of the blood vessel. To determine the parameters of this linear relationship (i.e. slope and bias), a calibration of the system is required, for example by determining the diastolic and systolic pressure, using e.g. a blood pressure cuff. Instead of assuming the arterial stiffness as a constant, the PWV can be used to determine the blood pressure and take into consideration the dependence of the arterial stiffness on the pressure. In this case, the blood pressure is a function of the PWV and the diameter change of the blood vessel. Using the PWV, the absolute pressure change, from diastolic to systolic pressure, can be determined without any calibration. However, the baseline pressure can then be added using a single pressure calibration. This disclosure provides example embodiments of automatic determination of the blood vessel wall diameter change of time and of the pulse wave velocity. Other blood vessel characteristics can then be determined based on these determinations, such as blood pressure, vascular distensibility, vascular compliance, and many others.

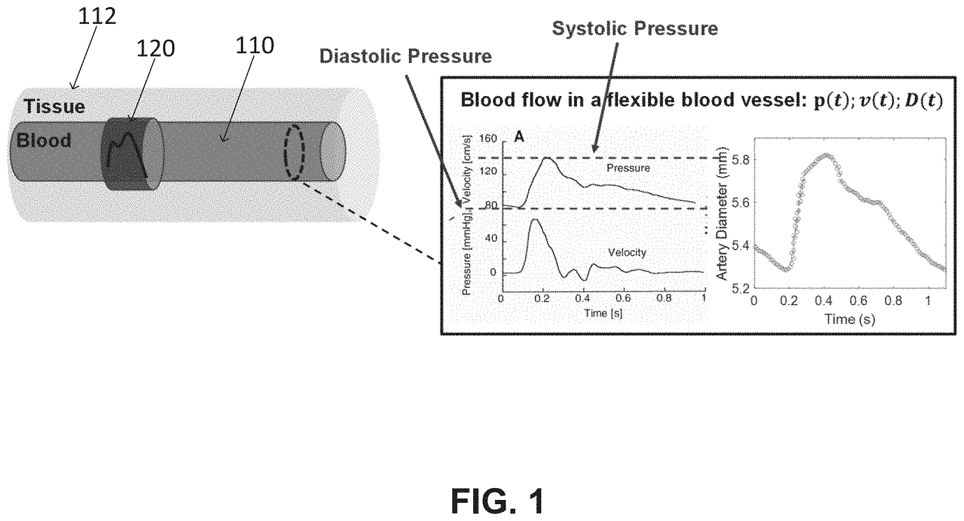

FIG. 1 illustrates a representation of a pressure wave 120 moving through a blood vessel 110, according to some embodiments. As illustrated, blood vessel 110 is surrounded by tissue 112 (e.g., fat or muscle). Embodiments of the system described herein can be used to measure the expansion wave 120 and blood flow and blood vessel characteristics. In some embodiments, the system is comprised within a wellness monitoring device, also referred to herein as a health sensor, which may be a handheld device or a wearable device (such as e.g. a watch).

As discussed above, in conventional medical ultrasonic imaging, first an image or plurality of images of the blood vessel would be captured, and then the required information is deduced from the image (sequence). In the described sensing system, the blood vessel 110 and blood flow characteristics are measured in a more direct method, automatically, without an intermediate imaging process, and without the requirement of a technician. The sensor transmits ultrasonic waves which are reflected at any boundaries in the tissue that have an acoustic impedance mismatch, e.g., the boundaries and walls of the blood vessels. The reflected signal also comes from ultrasound waves reflected inside the tissue through various scattering mechanisms, and the reflected signals are therefore not only from blood vessels. The reflected ultrasound waves are then measured by the sensor. The Time-Of-Flight (TOF) of the signal is an indication of the depth of the feature the signal reflected off. Using the received signals, blood vessel wall velocity caused by blood flow in a flexible blood vessel can be determined, allowing for the determination of various blood vessel characteristics, such as blood pressure (as illustrated by the Systolic pressure and the Diastolic pressure), blood vessel diameter change, and pulse wave velocity.

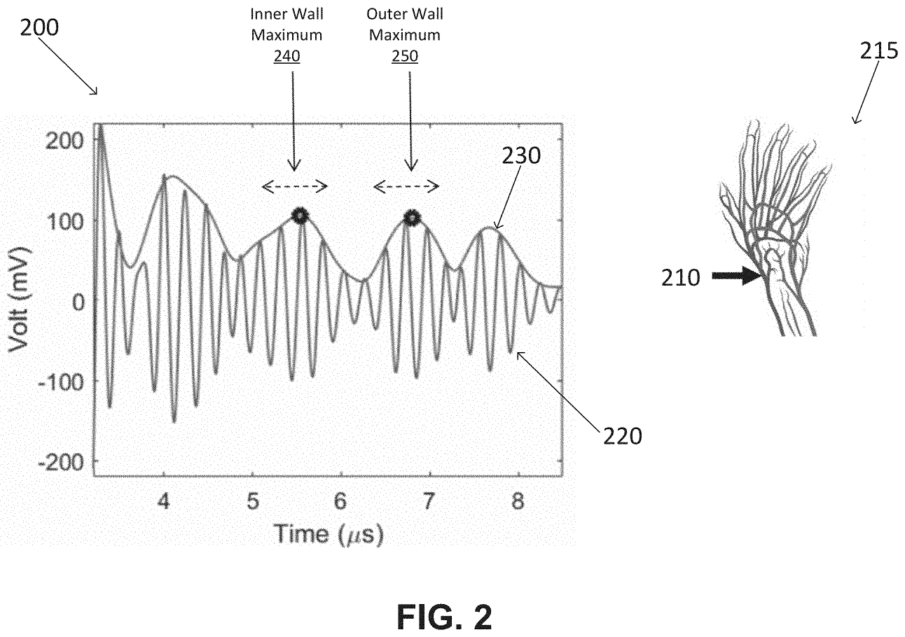

FIG. 2 illustrates an example graph 200 of measurements of the reflected signal for use in determining blood vessel wall depth, according to some embodiments. As illustrated, FIG. 2 shows example measurements from a blood vessel, such as radial artery 210 of hand 215. Graph 200 illustrates the ultrasonic signal waves 220 and the signal envelope 230. The signal envelope 230 shows local maxima that represent features in the hand where the waves reflected. Two of these maxima corresponds to the inner wall (e.g., inner wall maximum 240) and outer wall (e.g., outer wall maximum 250) of the blood vessel. The positions of these maxima 240 and 250, and the distance between the maxima, change over time because of the pressure/expansion wave with every heartbeat that changes the diameter of the blood vessel.

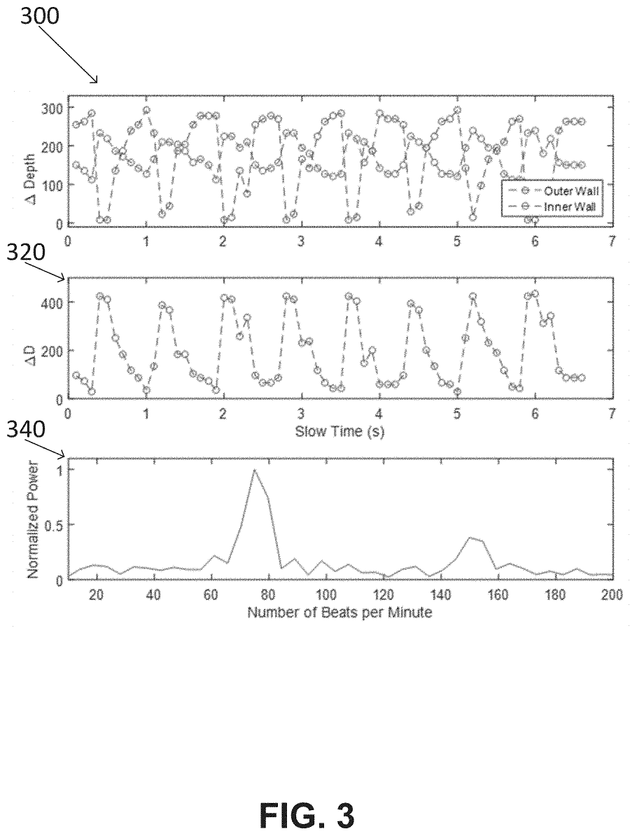

FIG. 3 illustrates example graphs of variations in relative position of blood vessel walls of a blood vessel over time, a change in diameter of the blood vessel over time, and heart beats per minute, according to some embodiments. Graph 300 shows the variations of the positions of the inner and outer wall as a function of time, graph 320 shows the change in vessel diameter based on the difference between the inner and outer wall, and graph 340 shows a frequency spectrum based on the measurements, which can be used to determine the heart rate. In this example, the peak at 75 beats/min represents the heart rate. Graph 340 also shows the higher order component at 150 Hz, which can also be used to characterize the cardiovascular system. The signature of the blood vessel characteristic may also be used for user authentication, since the blood flow patterns may differ from person to person. This authentication data may then be combined with other authentication methods, e.g., a fingerprint sensor. Although this example uses the radial artery in the hand, it should be appreciated that this can be applied at any artery or blood vessel at any place on the human body. The location on the body may be indicated or may be derived based on the ultrasound measurements (and compared to older measurements). Moreover, multiple sensors could be used across different body parts, at the same time or sequentially, for large area wellness parameters mapping. Communication of the sensor may be wired or wireless. When measuring different parts of the body at different times, synchronization of the data and timing of the sensors are performed for better analysis. Each sensor may emit a synchronization pulse, which may be an ultrasonic pulse emitted by the transducers, and a separate radio frequency (RF) pulse.

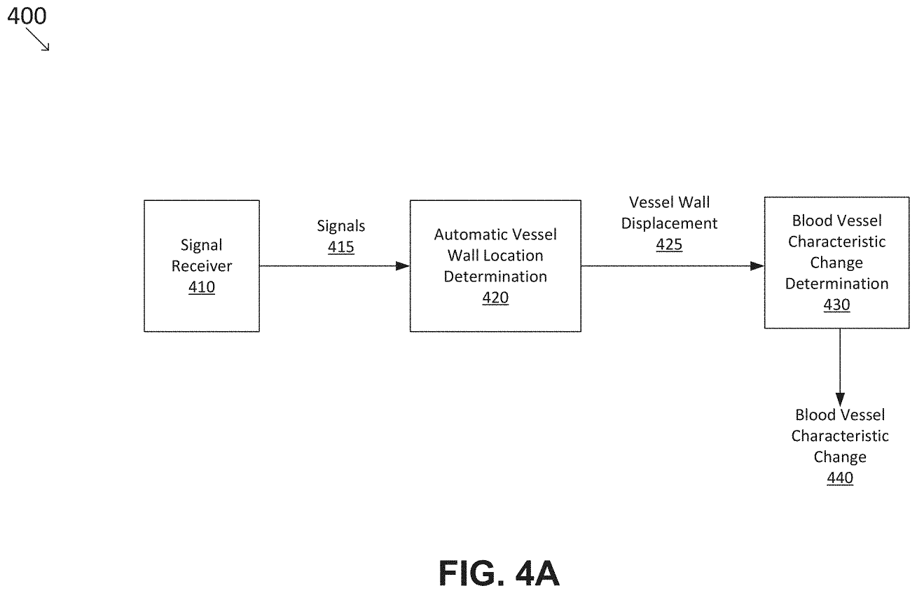

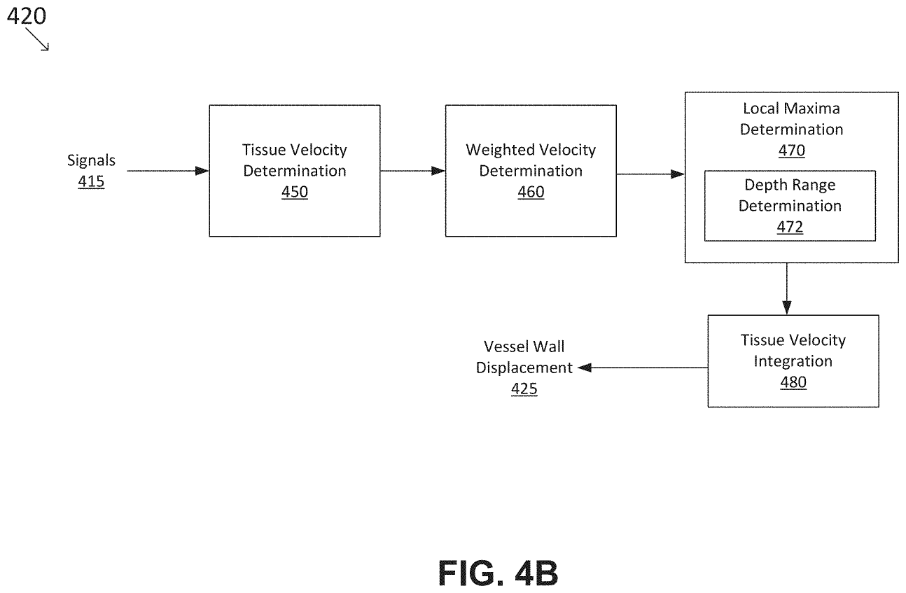

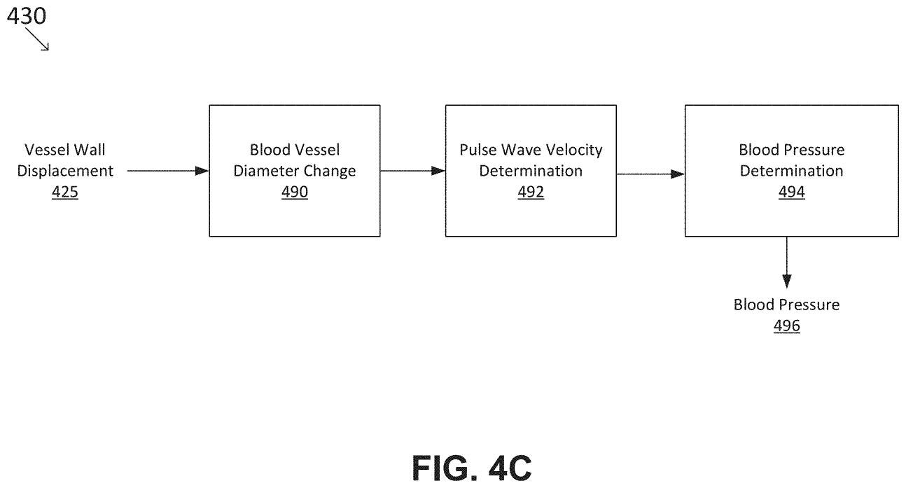

FIG. 4A illustrates a block diagram of an example system 400 for determining blood vessel characteristic (change) using an ultrasonic sensor, according to some embodiments. System 400 is configured to determine a change in a blood vessel characteristic based at least in part on signals (e.g., acoustic signals) received from an ultrasonic sensor. A sensing device is placed on the human body overlying a blood vessel at a stable location such that the ultrasonic sensor performs ultrasonic signal transmit and receive operations into the body tissue including a blood vessel. It should be appreciated that system 400 can be implemented as hardware, software, or any combination thereof. It should also be appreciated that signal receiver 410, automatic vessel wall location determination 420, and blood vessel characteristic change determination 430 may be separate components, may be comprised within a single component, or may be comprised in various combinations of multiple components, in accordance with some embodiments.

Ultrasonic signals are received at signal receiver 410. It should be appreciated that, in accordance with various embodiments, signal receiver 410 is an ultrasonic sensor (e.g., a sensor capable of transmitting and receiving ultrasonic signals) or coupled to an ultrasonic sensor. The ultrasonic sensor is operable to emit and detect ultrasonic waves (also referred to as ultrasonic signals or ultrasound signals). One or more ultrasonic transducers (e.g., Piezoelectric Micromachined Ultrasonic Transducers (PMUTs)), which may be comprised within an array configured to determine blood vessel measurement, may be used to transmit and receive the ultrasonic waves, where the ultrasonic transducers are capable of performing both the transmission and receipt of the ultrasonic waves. The emitted ultrasonic waves are reflected from any objects in contact with (or in front of) the ultrasonic sensor, and can project into the object at various depths, and these reflected ultrasonic waves, or echoes, are then detected. Where the object is a human body (e.g., at an arm or a wrist), the waves are projected into the tissue of the human body, and reflect at different tissue depths due to acoustic impedance mismatches.

Signal receiver 410 communicates signals 415 to automatic vessel wall location determination 420 which is configured to automatically determine depths of blood vessel walls at the position for a plurality of time instances based on local maxima of a combination of an acoustic impedance mismatch and a motion characteristic based at least in part on the received signals 415. An acoustic impedance mismatch happens at boundaries between materials having different acoustic properties, e.g., blood vessel walls and blood flowing in the blood vessel or the tissue around the blood vessels. In some embodiments, the motion characteristic is the velocity of the blood vessel walls as it expands and contracts, e.g., the velocity of the blood vessel walls moving away from and towards the ultrasonic sensor positioned on the body.

FIG. 4B illustrates a block diagram of an example automatic vessel wall location determination 420, according to some embodiments. Signals 415 are received at automatic vessel wall location determination 420. In some embodiments, automatic vessel wall location determination 420 is configured to determine the velocity of the tissue at the plurality of time instances based at least in part on the plurality of received signals using at least a phase of the received signals.

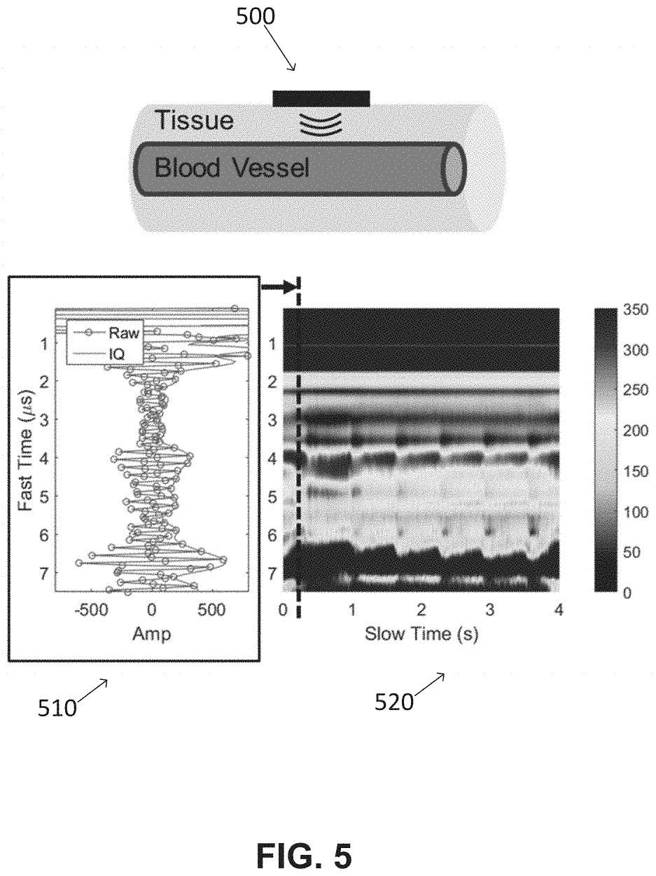

FIG. 5 illustrates an example fast time and slow time graphs of received signals for use in determining blood vessel wall depth using ultrasonic sensing system 500 positioned over tissue and a blood vessel, according to some embodiments. As utilized herein, fast time refers to microsecond (μs) scale of the time-of-flight of the ultrasound signal and slow time refers to the second (s) scale as a series of measurements is taken. Graph 510 illustrates the amplitude of the raw signal and the IQ signal on the fast time scale, and graph 520 illustrates the IQ signal cumulatively over the slow time scale, which represented that pulsating action of the blood vessel as it expands and contracts. These are signals 415 of FIGS. 4A and 4B.

With reference to FIG. 4B, signals 415 are received at tissue velocity determination 450 of automatic vessel wall location determination 420. Tissue velocity determination 450 is configured to determine the velocity of the tissue at the plurality of time instances and at different depths based at least in part on the plurality of received signals using at least a phase of the received signals. In one embodiment, the velocity is determined by performing Doppler signal processing on signals 415 to determine the velocity of the tissue at the plurality of time instances. For example, the velocity at each depth is derived using the phase difference between subsequent received reflected signals from that depth using conventional Doppler techniques.

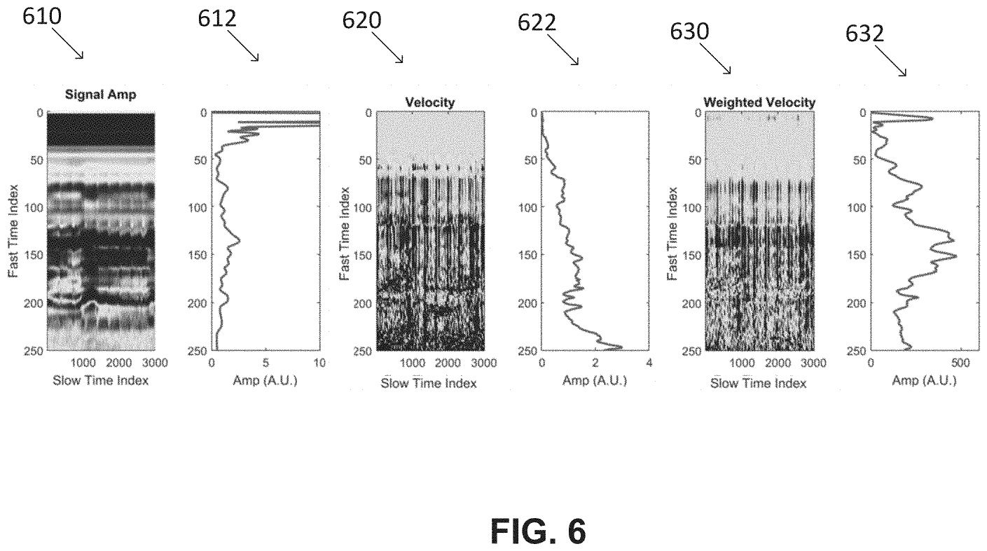

FIG. 6 illustrates example fast time and slow time graphs of signal strength, tissue velocity, and weight tissue velocity for use in determining blood vessel wall depth, according to some embodiments. The captured reflected ultrasound signal contains signal amplitude information and phase information. Graph 610 illustrates the amplitude component of signal 415 cumulatively over the slow time scale (e.g., similar to graph 520), where darker tones represent a higher reflected amplitude. Graph 612 illustrates the amplitude of signal 415 over the fast time scale (e.g., similar to graph 510) for multiple measurements. Similarly, graph 620 illustrates the phase component of the reflected signal, converted in a velocity measure, using, e.g., Doppler processing. Graph 622 shows a velocity profile as a function of the fast time (i.e. depth). The velocity is calculated based on phase, which is very sensitive to noise, such that a region of graph 620 with a low signal to noise ratio (SNR), e.g. the deeper layers, leads to a peak value of velocity and a large uncertainty.

The automatic detection of the location of the blood vessel is based on a combination of the following insights: 1) the blood vessel walls have a distinct acoustic impedance mismatch with the surrounding blood and tissue, and 2) the vessel walls move in a direction perpendicular to the blood flow. The described system takes advantage of these properties to automatically locate the blood vessel because the vessel walls have the highest combined impedance mismatch and tissue velocity (in the required direction). One example method to use a parameter to express the combined impedance mismatch and tissue velocity, is to introduce a weighted tissue velocity, where the weight is based on the impedance mismatch (e.g., the reflected signal intensity). The location of blood vessel walls is based on both an acoustic impedance mismatch, as indicated in signal 415, and the velocity of the tissue. With reference to FIG. 4B, in some embodiments, the velocity as determined at tissue velocity determination 450 is forwarded to weighted velocity determination 460 for determining a weighted velocity. A weighted velocity is generated based on the velocity and the received signals 415 (e.g., multiplying received signals 415 by the determined velocity). Weighted velocity determination 460 is configured to determine a weighted velocity of the tissue at the plurality of time instances based on signal amplitudes of the plurality of received signals at the plurality of time instances and depths and the velocity of the tissue at the plurality of time instances.

With reference to FIG. 6, graphs 630 and graphs 632 illustrate examples of using the signal amplitude to weight the calculated velocity, such that the region with real vessel motion is amplified. In other words, weighting the calculated velocity with the signal amplitude identifies the locations of peak vessel wall motion.

With reference to FIG. 4B, the weighted velocity is forwarded to local maxima determination 470. Local maxima determination 470 is configured to detect at least one local maximum of the combination of the acoustic impedance mismatch and the motion characteristic based at least in part on the plurality of received signals, wherein the at least one local maximum corresponds to a blood vessel wall. In some embodiments, local maxima determination 470 is configured to detect two local maxima correspond to the blood vessel walls. In some embodiment, verification of the correct location can be determined by the velocities at the local maxima being of the same or similar magnitude and in the opposite direction (e.g., out of phase).

In some embodiments, local maxima determination 470 includes depth range determination 472 which is configured to determine two depth ranges for the blood vessel based on blood vessel geometry. While the location and geometry of blood vessels can vary from person to person, it is possible to determine two depth ranges, where a first depth range includes a closer wall of the blood vessel relative to the ultrasonic sensor and a second depth range includes a farther wall of the blood vessel relative to the ultrasonic sensor. A first local maximum weighted velocity is determined within the first depth range, where the first local maximum weighted velocity within the first depth range corresponds to the depth of the closer wall of the blood vessel, and second local maximum weighted velocity is determined within the second depth range, where the second local maximum weighted velocity within the second depth range corresponds to the depth of the farther wall of the blood vessel. In some embodiments, the first depth range and second depth range can be determined during a calibration phase, and can be used for location verification using the approximate distance between the maxima.

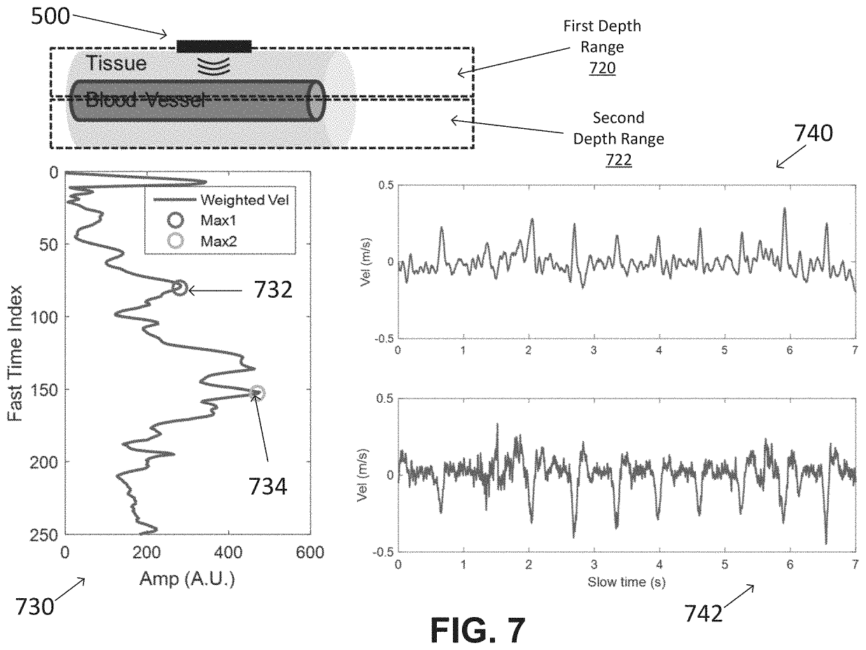

FIG. 7 illustrates example graphs for identifying local maxima of a combination of an acoustic impedance mismatch and a motion characteristic based the received ultrasonic signals for identifying the depth of the blood vessel walls relative to the ultrasonic sensor, according to some embodiments. FIG. 7 illustrates first depth range 720 and second depth range 722 relative to ultrasonic sensing system 500, where first depth range 720 includes a closer wall of the blood vessel relative to ultrasonic sensing system 500 and second depth range 722 includes a farther wall of the blood vessel relative to sensing system 500.

Graph 730 illustrates the weighted velocity (e.g., graph 632). Using first depth range 720 and second depth range 722, where the first depth range corresponds to 50-100 on the Fast Time Index and the second depth range corresponds to 125-175 on the Fast Time Index, two maximum velocities 732 and 734 can be determined, wherein each maximum weighted velocity is associated with a depth location of the corresponding blood vessel wall. Graphs 740 and 742 illustrate the velocity profiles at the two local maxima indicated in graph 730. In embodiments without predefined depth ranges, the local maxima in the weighted velocity graph can be used to find the blood vessel walls. A sliding windows may be used to find the local maxima, where the windows size is related to the (approximate) size of the blood vessel. As discussed above, additional criteria can be used to verify that the candidate locations of the blood vessel walls corresponding to the local maxima are indeed the walls of the blood vessel. Graph 730 shows how to use the weighted velocity to determine the vessel diameter (change). This can be done at a plurality of lateral locations to determine the center location and/or vessel geometry. Furthermore, a similar strategy as discussed to determine the vessel diameter can be used to determine the vessel wall thickness. In this case, the first impedance mismatch is between the tissue and the blood vessel wall, and the second impedance mismatch is between the vessel wall and the blood in the vessel. This would then again lead to two local maxima in the weighted velocity plots, where the distance between the maxima is a measure for the vessel wall thickness. Because of the smaller dimension of the vessel wall thickness compared to the vessel wall diameter, a higher resolution and accuracy (e.g., SNR) is required.

With reference to FIG. 4B, the local maxima determination and corresponding velocity information is forwarded to tissue velocity integration 480, which is configured to integrate the velocity of the blood vessel at the depth of the closer wall and the velocity of the blood vessel at the depth of the farther wall to generate a displacement of the closer wall and a displacement of the farther wall, collectively referred to as vessel wall displacement 425. Velocity is integrated to calculate displacement, where the difference of the two displacement of the upper and lower vessel walls leads to the absolute diameter change. In addition, by using the TOF between the vessel walls to determine the absolute diameter of the blood vessel, the variation in the absolute blood vessel diameter can be determined over time.

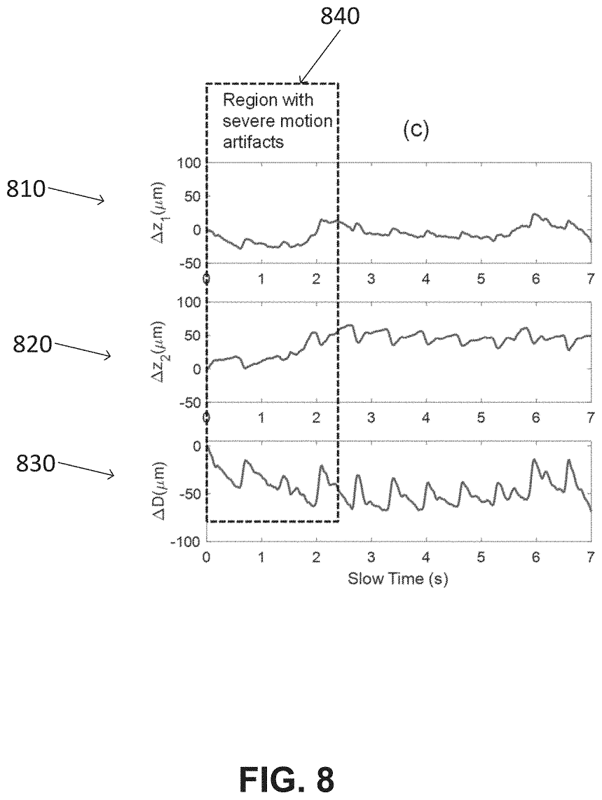

FIG. 8 illustrates example graphs of displacement of the blood vessel walls and the change in diameter of the blood vessel over time, according to some embodiments. Graphs 810 and 820 illustrate the result of integrating the velocity profiles (e.g., graph 740 and 742), generating the depth change of the blood vessel walls relative the ultrasonic sensor over time. Graph 830 illustrates the diameter change of the blood vessel over time, which is the difference of the two displacement of the blood vessel walls of graphs 810 and 820. In order to determine the absolute blood vessel diameter, the TOF can be used between the local maxima of the weighted velocity, as seen for example in graph 730. For example, the average TOF between the vessel walls may be determined and combined with the speed of sound to obtain the average absolute vessel diameter. The absolute diameter change (e.g., of graph 830) is then around this average absolute vessel diameter.

With reference to FIG. 4A, automatic vessel wall location determination 420 forwards vessel wall depth 425 to blood vessel characteristic change determination 430. Blood vessel characteristic (change) determination 430 is configured to determine a blood vessel characteristic change 440 based at least in part on a difference between the depths of the blood vessel walls at the plurality of time instances. In some embodiments, vessel wall depth 425 includes the diameter change of the blood vessel over time. In some embodiments, blood vessel characteristic change determination 430 is configured to determine the diameter change of the blood vessel over time.

Based on the ultrasound measurements, different characteristics of the blood vessel and blood flow can be determined. Furthermore, by monitoring the characteristics over time changes in the characteristics or wellness of the user may be determined. The data and results of the sensor may also be combined with results from other sensors such as an ECG or PPG. The data of the sensor may also be linked to the context and/or activities of the user to monitor the wellness of the user in relation to the context and/or activities. The wellness device containing the sensor may also include other sensors, e.g., motion sensors, for determine the context and/or activity.

In some embodiments, blood vessel characteristic change determination 430 is configured to determine a blood pressure using vessel wall displacement 425. In some embodiments, blood vessel characteristic change determination 430 is configured to determine a diameter change of the blood vessel using vessel wall displacement 425.

FIG. 4C illustrates a block diagram of an example blood vessel characteristic change determination 430, according to some embodiments. In the illustrated embodiment, blood vessel characteristic change determination 430 is configured for determining blood pressure. Vessel wall displacement 425 is received at blood vessel diameter change 490, which is configured to determine the change in blood vessel diameter. Pulse wave velocity determination 492 is configured to determine the pulse wave velocity. Using the blood vessel diameter change and the pulse wave velocity, blood pressure determination 494 is configured to determine blood pressure 496. As discussed above, the blood vessel geometry and material properties determine the correlation between the diameter change to blood pressure change. Assuming this relationship is linear, the diameter change can be calibrated to obtain the blood pressure change. The calibration can be done with conventional blood pressure cuff. On other embodiments, pulse wave velocity is a function of blood vessel geometry and material properties and can be measured to obtain the correlation between the diameter change to blood pressure change without any assumption or calibration. Pulse wave velocity is obtained via tracking the speed of the pressure wave propagating along the blood vessel wall. Along the vessel wall, the waveforms of vessel wall displacement, velocity, or acceleration (derivative of vessel wall velocity over time) can be recorded. Then the timing for the occurrence of the same feature (such as maximum in amplitude) along the vessel walls can be used to calculate the time for the pressure wave to travel from one segment of the vessel wall to another. The vessel length can be measured using ultrasonic transducer arrays. The pulse wave velocity can then be calculated using the vessel length divided by the time of travel.

With reference to FIG. 8, in some operating conditions, motion artifacts, some of which might be severe, are present in graphs 810 and 820 of the velocity profiles. These motion artifacts may be caused by movement of the sensing system relative to the placement on the body during transmission and receipt of the ultrasonic signals. As shown in region 840, some of the motion artifacts on the blood vessel walls are in phase and naturally cancel out with subtraction, such that region 840 of graph 830 exhibits less motion artifact change.

In some instances, the motion artifacts may be so severe that the diameter calculation does not naturally reduce or cancel out the motion artifacts. In some embodiments, a motion characteristic (e.g., velocity) is determined at tissue outside the blood vessel and used to correct for motion artifacts. For example, the velocity within a stationary layer of tissue between the blood vessel and the ultrasonic sensor can be determined. Since this stationary layer is not, or less, influenced by the vessel motion, and detected motion is linked to external motion that can cause motion artifacts, the determined motion at the stationary layer can then be used to correct the determined vessel wall motion for any motion artifacts due to external motion.

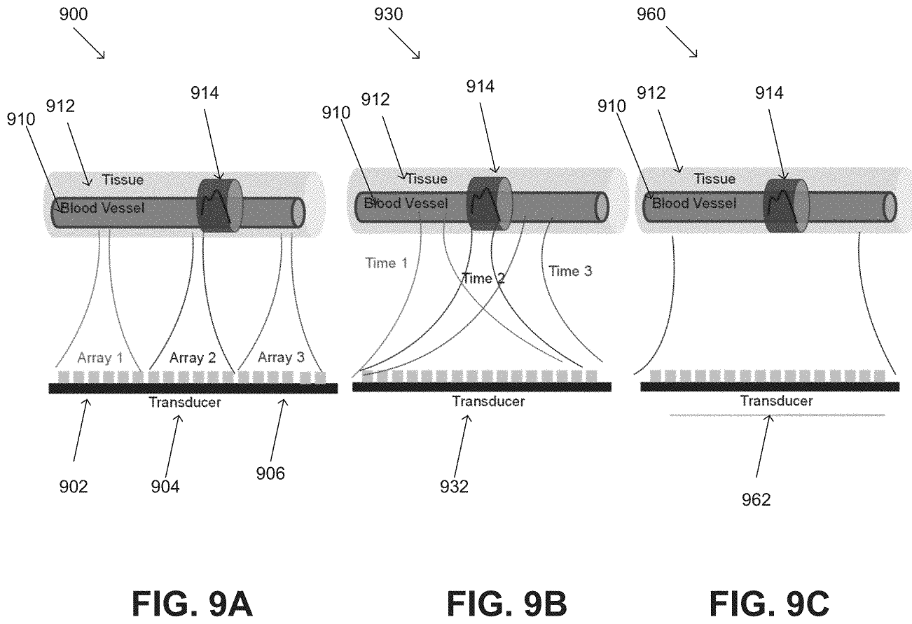

FIGS. 9A, 9B, and 9C illustrate different embodiments of a wellness sensing system for extracting pulse wave velocity information. As discussed above, the pulse wave velocity represents the speed with which the blood vessel expansion propagates along the blood vessel. To determine the pulse wave velocity, ultrasound measurements along the blood vessel are performed. The measurement can be performed according to different methods and sensor configurations. FIG. 9A illustrates an example wellness sensing system 900 including multiple focused acoustic arrays 902, 904, and 906 for performing blood vessel characteristic change determination in a synchronized order, where each array 902, 904, and 906 focuses on a different portion of blood vessel 910 within tissue 912 for identifying the motion of pressure wave 914. The different arrays can be part of the same sensor, or can be separate sensors. FIG. 9B illustrates an example wellness sensing system 930 including linear array 932 for performing blood vessel characteristic change determination by focusing the ultrasonic beam on different locations of blood vessel 910 within tissue 912 at different times during a multiple stage signal acquisition for identifying the motion of pressure wave 914. FIG. 9C illustrates an example wellness sensing system 960 including array 962 using a large plane wave over the full array 962 to perform signal acquisition on blood vessel 910 within tissue 912 during signal acquisition for identifying pressure wave 914. A plane wave is transmitted continuously. In each transmission, a snapshot of the blood vessel is reconstructed. The location of the pressure wave between snapshots can give distance traveled, while the difference in slow time gives the time. The pulse wave velocity can be then be calculated as distance divided by time.

For each of the configurations illustrated in FIGS. 9A, 9B, and 9C, the same blood vessel characteristics may be determined at the different positions, and these results may then be measured. The timing difference between the blood vessel expansions at the different locations can then be used to determine the pulse wave velocity. Synchronization of the timing between the different array/beams is required for an accurate pulse wave velocity determination. In some embodiments, a detailed blood vessel characteristic may be determined only at limit number of locations (e.g. not all, but only one location), for example only at one array or using one beam, while the other arrays or beams are used to determine the pulse wave velocity. Multiple arrays increase the examined area and add redundant measurements to accurately extract the blood vessel characteristics, which reduce the sensor alignment requirement.

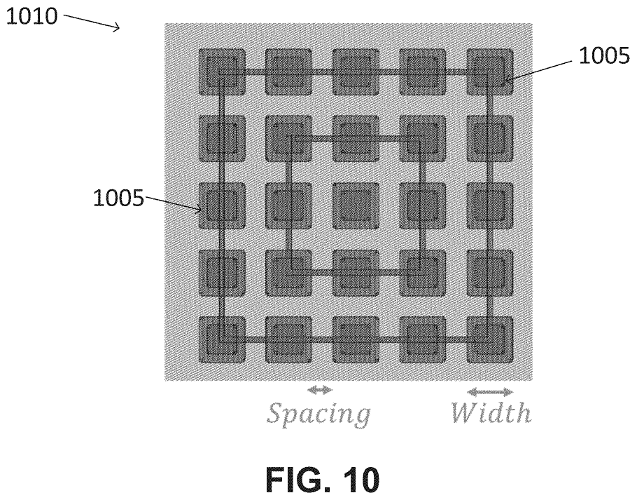

FIG. 10 illustrates an example layout of ultrasonic transducers 1005 of an ultrasonic sensor 1010, according to an embodiment. In this example, ultrasonic sensor 1010 comprises an array of ultrasonic transducers 1005. However, other principles of ultrasonic sensors using bulk piezoelectric or film based piezoelectric materials may also be used. FIG. 10 shows a 5×5 array of ultrasonic transducers 1005. This array is just an example, and more or fewer transducers may be used, and the array may have other form factors (e.g., a linear array). Each of these transducers may be a Piezoelectric Micromachine Ultrasonic Transducer (PMUT), fabricated using e.g. MEMS technologies. It should be appreciated other layouts and configurations of ultrasonic transducers can be used, of which FIG. 10 is one example.

The array of transducers may be used for forming and steering an ultrasonic beam. The beam forming can be used to focus the ultrasonic waves at the correct depth, and the beam steering may be used to control lateral motion of the beam to find the blood vessel. For example, when the sensor is placed on the skin, the sensor may not be exactly above the blood vessel. The beam steering and beamforming may be used to find the vessel in a first step through a scanning action, and once the vessel is located, in a second step perform the blood vessel and blood flow measurements. The beam forming and beam steering can be accomplished by applying small phase delays to the individual transducers. The PMUTs may be controlled individually, or the PMUTs may be grouped together in subsets of PMUTs. These subset of pixels may be connected together. For example, FIG. 10 shows the array of transducers is divided into three subsets; the outer ring of transducers, the middle ring of transducers, and the center transducer. This type of layout helps with beamforming around the center of the sensor. Other subgroups can be used for other type of beam forming and beam steering, for example by forming subset of rows or columns of transducers. This may be done for generating the ultrasonic beam (transmit beamforming), but it may also help with the signal analysis of the detected reflected waves (receive beamforming). Location of the blood vessel may also be based on Doppler measurements or by looking for signal with the right heartbeat signal or frequency components. Furthermore, optimizing for a maximum change in amplitude can be used to determine the center middle of the blood vessel. The system can be a closed loop system meaning it will adapt operational parameters of the sensor autonomously to obtain the best results. The operational parameters include settings for the beam forming and steering or any other parameters related to the transmit and/or receive functions of the sensor.

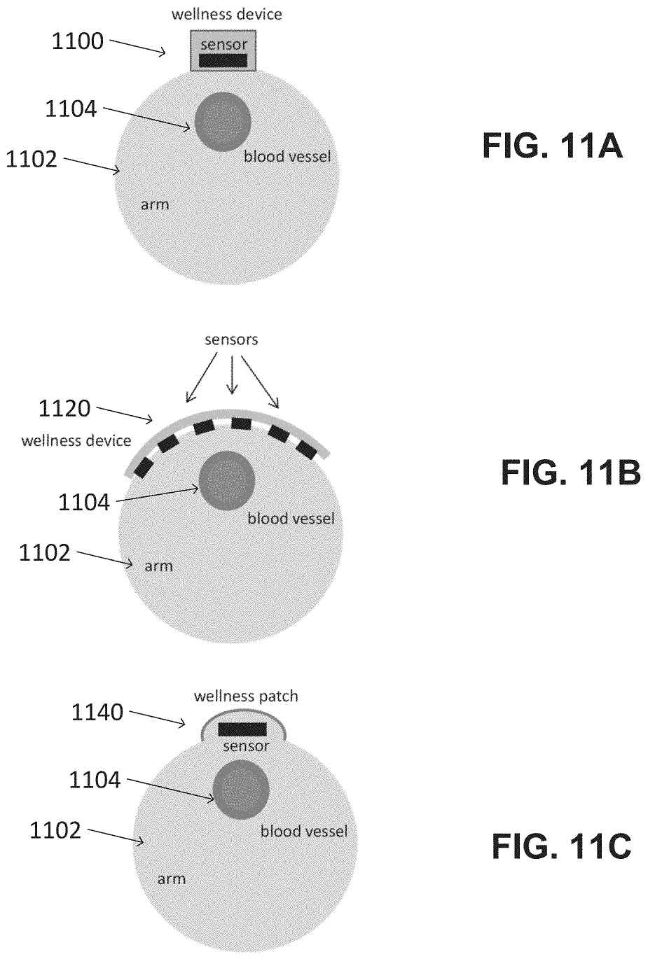

FIGS. 11A, 11B, and 11C illustrate different examples of wellness devices having an ultrasonic sensor for determining blood vessel characteristic change; according to some embodiments. The wellness devices 1100, 1120, and 1140 described herein may include a single sensor or a plurality of sensors. As illustrated, wellness devices 1100, 1120, and 1140 are placed on arm 1102 and overlie blood vessel 1104 (neither of which is to scale and are for illustrative purposes). However, it should be appreciated that wellness devices 1100, 1120, and 1140 can be placed anywhere on the human body, subject to the arrangement and design for placement over a blood vessel.

FIG. 11A shows an example embodiment where the device 1100 contains a single sensor, while FIG. 11B shows an example embodiment where the device 1120 contains a plurality of sensors. The plurality of sensors may be rigidly connected, or may be connected in a flexible manner to follow the contours of the body where the measurements are taken. This means that the substrate and/or packaging of the sensor may be rigid or flexible depending on the application and device. The sensors may be incorporated for example in a blood measurement cuff or and armband (e.g., of a watch). The plurality of sensors may be organized in a one-dimensional array or a two-dimensional array, or any other organization required for the application. The sensors may also be part of a network of sensors place at different locations. Other sensors may be incorporated with the ultrasonic sensors, and may help determine the position and or shape of the array. The other sensors may be motion sensors, e.g., an accelerometer, or pressures sensor, optical sensors, etc. FIG. 11C shows an embodiment where the wellness device 1140 is a patch including an ultrasonic sensor that can be put on the skin of the user. The patch may have adhesive for staying put on the skin. The sensor may also have a contact surface to improve conduction of the ultrasound waves into the skin of the user. The contact surface may comprise a gel like compartment, or other material, to increase the acoustic coupling. The compartment may be designed for slow diffusion of an agent to increase the acoustic coupling. The patch may be completely autonomous and comprise sensor, processor, memory, and a battery for power. The data may be transmitted during operation and use, or stored for reading after use.

In some embodiments, the wellness devices may include additional sensors and/or actuators that work together with the ultrasonic sensor. For example, in a system like a blood pressure cuff, actuators may be used to press or inflate the cuffs, and a pressure sensor may be present for monitoring this process. The system may control the sensor based on the actuator or pressure sensor readings (or vice-versa). As a result, the sensor may provide cardiovascular data as a function of the applied pressure. The principle of applying different pressures or forces may also enable characterization that would not be possible at a static situation. Other combinations of sensors and actuators are also envisioned for various applications.

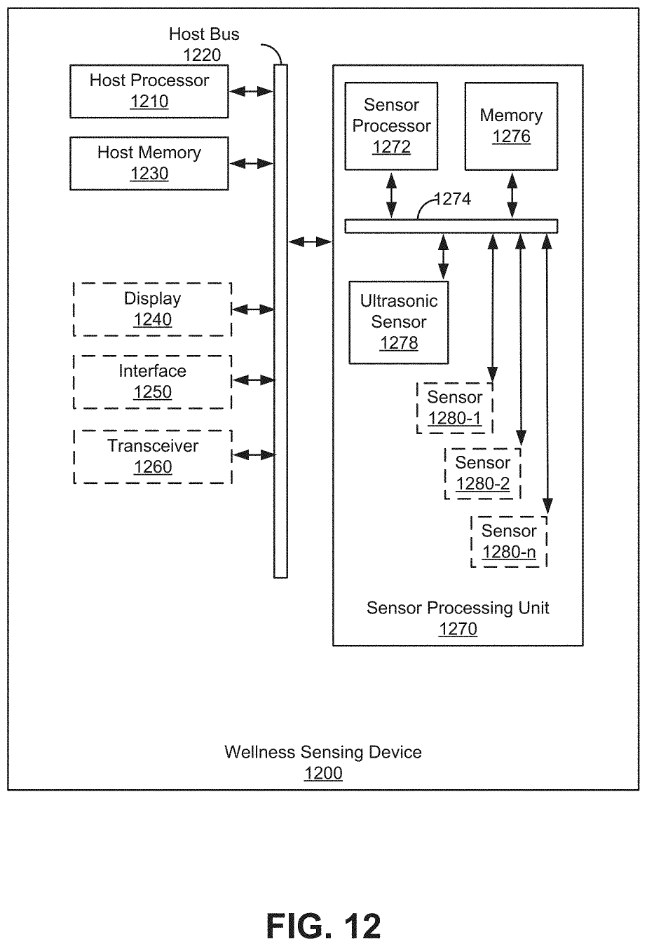

Turning now to the figures, FIG. 12 is a block diagram of an example wellness sensing device 1200. As will be appreciated, wellness sensing device 1200 may be implemented as a device or apparatus, such as a handheld mobile electronic device or a wearable device such as an activity or fitness tracker device (e.g., bracelet, clip, band, or pendant), a smart watch or other wearable device, or a combination of one or more of these devices. In accordance with various embodiments, wellness sensing device 1200 is capable of determining a blood vessel characteristic change.

As depicted in FIG. 12, wellness sensing device 1200 may include a host processor 1210, a host bus 1220, a host memory 1230, and a sensor processing unit 1270. Some embodiments of wellness sensing device 1200 may further include one or more of a display device 1240, an interface 1250, a transceiver 1260 (all depicted in dashed lines) and/or other components. In various embodiments, electrical power for wellness sensing device 1200 is provided by a mobile power source such as a battery (not shown), when not being actively charged.

Host processor 1210 can be one or more microprocessors, central processing units (CPUs), DSPs, general purpose microprocessors, ASICs, ASIPs, FPGAs or other processors which run software programs or applications, which may be stored in host memory 1230, associated with the functions and capabilities of wellness sensing device 1200.

Host bus 1220 may be any suitable bus or interface to include, without limitation, a peripheral component interconnect express (PCIe) bus, a universal serial bus (USB), a universal asynchronous receiver/transmitter (UART) serial bus, a suitable advanced microcontroller bus architecture (AMBA) interface, an Inter-Integrated Circuit (I2C) bus, a serial digital input output (SDIO) bus, a serial peripheral interface (SPI) or other equivalent. In the embodiment shown, host processor 1210, host memory 1230, display 1240, interface 1250, transceiver 1260, sensor processing unit (SPU) 1270, and other components of wellness sensing device 1200 may be coupled communicatively through host bus 1220 in order to exchange commands and data. Depending on the architecture, different bus configurations may be employed as desired. For example, additional buses may be used to couple the various components of wellness sensing device 1200, such as by using a dedicated bus between host processor 1210 and memory 1230.

Host memory 1230 can be any suitable type of memory, including but not limited to electronic memory (e.g., read only memory (ROM), random access memory, or other electronic memory), hard disk, optical disk, or some combination thereof. Multiple layers of software can be stored in host memory 1230 for use with/operation upon host processor 1210. For example, an operating system layer can be provided for wellness sensing device 1200 to control and manage system resources in real time, enable functions of application software and other layers, and interface application programs with other software and functions of wellness sensing device 1200. Similarly, a user experience system layer may operate upon or be facilitated by the operating system. The user experience system may comprise one or more software application programs such as menu navigation software, games, device function control, gesture recognition, image processing or adjusting, voice recognition, navigation software, communications software (such as telephony or wireless local area network (WLAN) software), and/or any of a wide variety of other software and functional interfaces for interaction with the user can be provided. In some embodiments, multiple different applications can be provided on a single wellness sensing device 1200, and in some of those embodiments, multiple applications can run simultaneously as part of the user experience system. In some embodiments, the user experience system, operating system, and/or the host processor 1210 may operate in a low-power mode (e.g., a sleep mode) where very few instructions are processed. Such a low-power mode may utilize only a small fraction of the processing power of a full-power mode (e.g., an awake mode) of the host processor 1210.

Display 1240, when included, may be a liquid crystal device, (organic) light emitting diode device, or other display device suitable for creating and visibly depicting graphic images and/or alphanumeric characters recognizable to a user. Display 1240 may be configured to output images viewable by the user and may additionally or alternatively function as a viewfinder for camera. It should be appreciated that display 1240 is optional, as various electronic devices, such as electronic locks, doorknobs, car start buttons, etc., may not require a display device.

Interface 1250, when included, can be any of a variety of different devices providing input and/or output to a user, such as audio speakers, touch screen, real or virtual buttons, joystick, slider, knob, printer, scanner, computer network I/O device, other connected peripherals and the like.

Transceiver 1260, when included, may be one or more of a wired or wireless transceiver which facilitates receipt of data at wellness sensing device 1200 from an external transmission source and transmission of data from wellness sensing device 1200 to an external recipient. By way of example, and not of limitation, in various embodiments, transceiver 1260 comprises one or more of: a cellular transceiver, a wireless local area network transceiver (e.g., a transceiver compliant with one or more Institute of Electrical and Electronics Engineers (IEEE) 802.11 specifications for wireless local area network communication), a wireless personal area network transceiver (e.g., a transceiver compliant with one or more IEEE 802.15 specifications for wireless personal area network communication), and a wired a serial transceiver (e.g., a universal serial bus for wired communication).

Wellness sensing device 1200 also includes a general purpose sensor assembly in the form of integrated Sensor Processing Unit (SPU) 1270 which includes sensor processor 1272, memory 1276, a ultrasonic sensor 1278, and a bus 1274 for facilitating communication between these and other components of SPU 1270. In some embodiments, SPU 1270 may include at least one additional sensor 1280 (shown as sensor 1280-1, 1280-2, . . . 1280-n) communicatively coupled to bus 1274. In some embodiments, at least one additional sensor 1280 is a force or pressure sensor (e.g. a touch sensor) configured to determine a force or pressure or a temperature sensor configured to determine a temperature at wellness sensing device 1200. The force or pressure sensor may be disposed within, under, or adjacent ultrasonic sensor 1278. In some embodiments, all of the components illustrated in SPU 1270 may be embodied on a single integrated circuit. It should be appreciated that SPU 1270 may be manufactured as a stand-alone unit (e.g., an integrated circuit), that may exist separately from a larger electronic device and is coupled to host bus 1220 through an interface (not shown). It should be appreciated that, in accordance with some embodiments, that SPU 1270 can operate independent of host processor 1210 and host memory 1230 using sensor processor 1272 and memory 1276.

Sensor processor 1272 can be one or more microprocessors, CPUs, DSPs, general purpose microprocessors, ASICs, ASIPs, FPGAs or other processors which run software programs, which may be stored in memory 1276, associated with the functions of SPU 1270. It should also be appreciated that ultrasonic sensor 1278 and additional sensor 1280, when included, may also utilize processing and memory provided by other components of wellness sensing device 1200, e.g., host processor 1210 and host memory 1230.

Bus 1274 may be any suitable bus or interface to include, without limitation, a peripheral component interconnect express (PCIe) bus, a universal serial bus (USB), a universal asynchronous receiver/transmitter (UART) serial bus, a suitable advanced microcontroller bus architecture (AMBA) interface, an Inter-Integrated Circuit (I2C) bus, a serial digital input output (SDIO) bus, a serial peripheral interface (SPI) or other equivalent. Depending on the architecture, different bus configurations may be employed as desired. In the embodiment shown, sensor processor 1272, memory 1276, ultrasonic sensor 1278, and other components of SPU 1270 may be communicatively coupled through bus 1274 in order to exchange data.

Memory 1276 can be any suitable type of memory, including but not limited to electronic memory (e.g., read only memory (ROM), random access memory, or other electronic memory). Memory 1276 may store algorithms or routines or other instructions for processing data received from ultrasonic sensor 1278 and/or one or more sensor 1280, as well as the received data either in its raw form or after some processing. Such algorithms and routines may be implemented by sensor processor 1272 and/or by logic or processing capabilities included in ultrasonic sensor 1278 and/or sensor 1280.

A sensor 1280 may comprise, without limitation: a temperature sensor, a humidity sensor, an atmospheric pressure sensor, an infrared sensor, a radio frequency sensor, a navigation satellite system sensor (such as a global positioning system receiver), an acoustic sensor (e.g., a microphone), an inertial or motion sensor (e.g., a gyroscope, accelerometer, or magnetometer) for measuring the orientation or motion of the sensor in space, or other type of sensor for measuring other physical or environmental factors. In one example, sensor 1280-1 may comprise an acoustic sensor, sensor 1280-2 may comprise a temperature sensor, and sensor 1280-n may comprise a motion sensor.

In some embodiments, ultrasonic sensor 1278 and/or one or more sensors 1280 may be implemented using a microelectromechanical system (MEMS) that is integrated with sensor processor 1272 and one or more other components of SPU 1270 in a single chip or package. Although depicted as being included within SPU 1270, one, some, or all of ultrasonic sensor 1278 and/or one or more sensors 1280 may be disposed externally to SPU 1270 in various embodiments.

The ultrasonic sensor 1278 may be used to obtain blood vessel and blood flow characteristics, and the ultrasonic sensor 1278 or SPU 1270 may transfer this data to the host device. The host processor 1210 may then convert the data into a wellness indicator, or may present the data to the user. The host device may contain different wellness sensors for measuring different health indicators. These sensors may be based on ultrasonic sensors, or other type of sensors (e.g., sensors 1280). The ultrasonic sensor 1278 may perform different types of characterizations, for example in different modes. In the discussion above, the focus was on blood flow measurements, but other measurements may be performed. For example, the ultrasonic sensor 1278 may measure tissue characteristics based on the reflected ultrasound waves and use that information to derive a health indicator.

### Example Operations for Operating an Ultrasonic Sensor for Automatic Determination of a Blood Vessel Characteristic Change

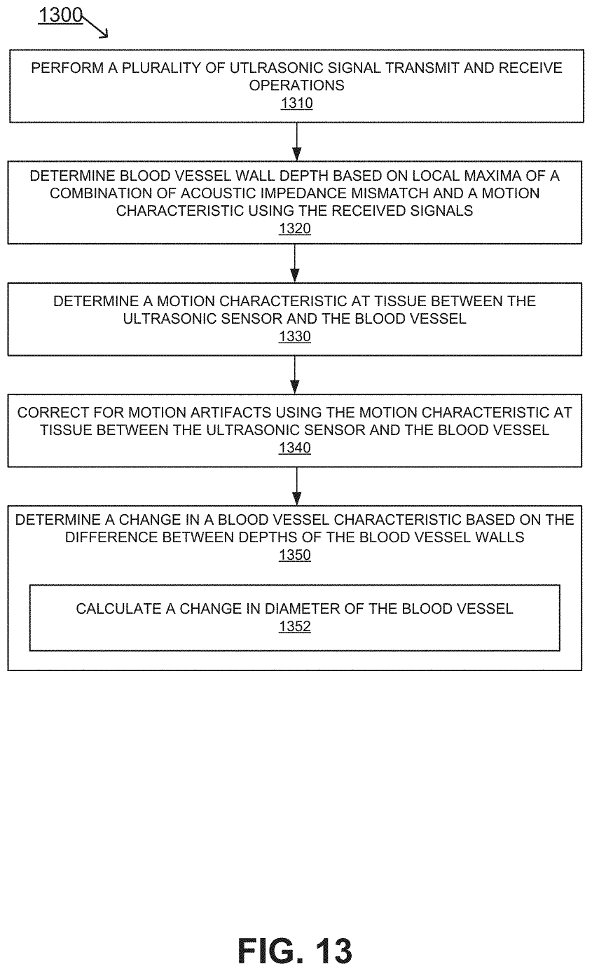

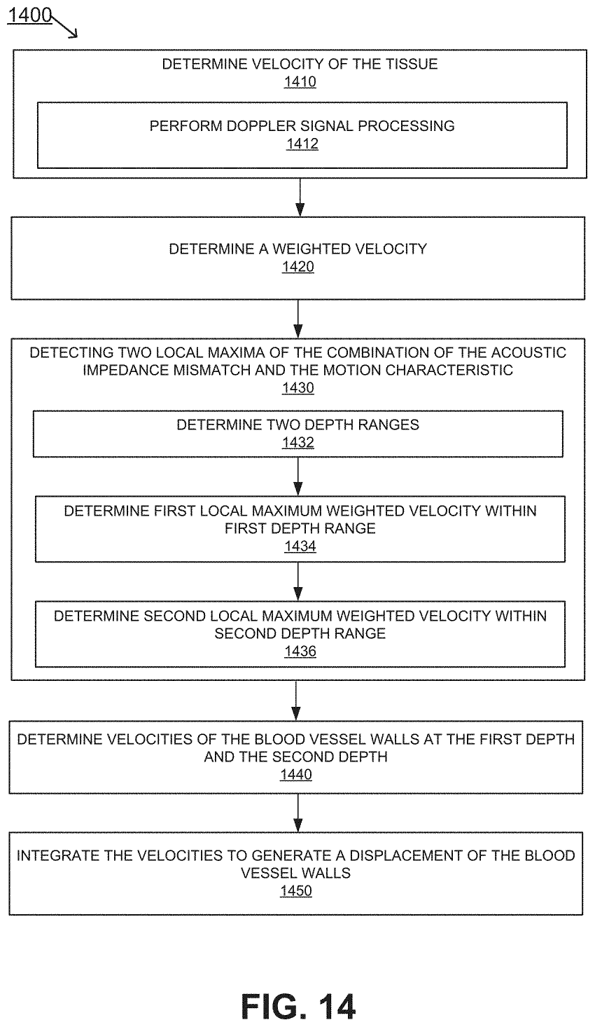

FIGS. 13 and 14 illustrate flow diagrams of example methods for determining blood vessel characteristic change using an ultrasonic sensor, according to various embodiments. Procedures of these methods will be described with reference to elements and/or components of various figures described herein. It is appreciated that in some embodiments, the procedures may be performed in a different order than described, that some of the described procedures may not be performed, and/or that one or more additional procedures to those described may be performed. The flow diagrams include some procedures that, in various embodiments, are carried out by one or more processors (e.g., a host processor or a sensor processor) under the control of computer-readable and computer-executable instructions that are stored on non-transitory computer-readable storage media. It is further appreciated that one or more procedures described in the flow diagrams may be implemented in hardware, or a combination of hardware with firmware and/or software.

With reference to FIG. 13, flow diagram 1300 illustrates an example process for determining blood vessel characteristic change using an ultrasonic sensor, according to some embodiments. At procedure 1310 of flow diagram 1300, a plurality of ultrasonic signal transmit and receive operations is performed at a position overlying a blood vessel of a person using an ultrasonic sensor, wherein the plurality of ultrasonic signal transmit and receive operations generate a plurality of received signals.

At procedure 1320, depths of blood vessel walls (e.g., a closer blood vessel wall and a farther blood vessel wall relative to the ultrasonic sensor) are automatically determined at the position for a plurality of time instances based on local maxima of a combination of an acoustic impedance mismatch and a motion characteristic based at least in part on the plurality of received signals.

In some embodiments, procedure 1320 is performed according to the procedures of flow diagram 1400 of FIG. 14. Flow diagram 1400 illustrates an example process for determining blood vessel wall depth, according to some embodiments. At procedure 1410 of flow diagram 1400, where the motion characteristic is a velocity of tissue, determination of the depths of blood vessel walls at the position for a plurality of time instances based at least in part on the plurality of received signals includes determining the velocity of the tissue at the plurality of time instances based at least in part on the plurality of received signals using at least a phase of the received signals. In one embodiment, as shown at procedure 1412, determination of the velocity of the tissue at the plurality of time instances includes performing Doppler signal processing on the plurality of received signals to determine the velocity of the tissue at the plurality of time instances.

At procedure 1420, a weighted velocity of the tissue at the plurality of time instances is determined based on signal amplitudes of the plurality of received signals at the plurality of time instances and the velocity of the tissue at the plurality of time instances. In one embodiment, the weighted velocity of the tissue depends on an impact of the acoustic impedance mismatch on the velocity of the tissue.

At procedure 1430, two local maxima of the combination of the acoustic impedance mismatch and the motion characteristic are determined based at least in part on the plurality of received signals, wherein the two local maxima correspond to the blood vessel walls. In some embodiments, as shown at procedure 1432, two depth ranges for the blood vessel based are determined on blood vessel geometry, where a first depth range comprises a closer wall of the blood vessel relative to the ultrasonic sensor and a second depth range comprises a farther wall of the blood vessel relative to the ultrasonic sensor. At procedure 1434, a first local maximum weighted velocity within the first depth range is determined, wherein the first local maximum weighted velocity within the first depth range corresponds to the depth of the closer wall of the blood vessel. At procedure 1436, a second local maximum weighted velocity within the second depth range is determined, wherein the second local maximum weighted velocity within the second depth range corresponds to the depth of the farther wall of the blood vessel. In one embodiment, the blood vessel characteristic is a change in blood vessel diameter.

At procedure 1440, a velocity of the blood vessel at the depth of the closer wall and a velocity of the blood vessel at the depth of the farther wall at the is determined plurality of time instances, where the velocity of the blood vessel at the depth of the closer wall and the velocity of the blood vessel at the depth of the farther wall are out of phase. At procedure 1450, the velocity of the blood vessel at the depth of the closer wall and the velocity of the blood vessel at the depth of the farther wall is integrated to generate a displacement of the closer wall and a displacement of the farther wall.

With reference to FIG. 13, in some embodiments, as shown at procedure 1330, the motion characteristic at tissue at a depth between the ultrasonic sensor and a closer blood vessel wall relative to the ultrasonic sensor is determined. In some embodiments, as shown at procedure 1340, motion artifacts within displacement of the blood vessel walls are corrected for by using the motion characteristic at tissue at a depth between the ultrasonic sensor and a closer blood vessel wall relative to the ultrasonic sensor.

At procedure 1350, a change in a blood vessel characteristic is determined based at least in part on a difference between the depths of the blood vessel walls at the plurality of time instances. In one embodiment, as shown at procedure 1352, a diameter of the blood vessel is calculated at the plurality of time instances based on a difference of the displacement of the closer wall and the displacement of the farther wall. In one embodiment, the blood vessel characteristic is a blood pressure. In another embodiment, the blood vessel characteristic is a pulse wave velocity of the blood vessel.

### CONCLUSION

The examples set forth herein were presented in order to best explain, to describe particular applications, and to thereby enable those skilled in the art to make and use embodiments of the described examples. However, those skilled in the art will recognize that the foregoing description and examples have been presented for the purposes of illustration and example only. Many aspects of the different example embodiments that are described above can be combined into new embodiments. The description as set forth is not intended to be exhaustive or to limit the embodiments to the precise form disclosed. Rather, the specific features and acts described above are disclosed as example forms of implementing the claims.

Reference throughout this document to “one embodiment,” “certain embodiments,” “an embodiment,” “various embodiments,” “some embodiments,” or similar term means that a particular feature, structure, or characteristic described in connection with the embodiment is included in at least one embodiment. Thus, the appearances of such phrases in various places throughout this specification are not necessarily all referring to the same embodiment. Furthermore, the particular features, structures, or characteristics of any embodiment may be combined in any suitable manner with one or more other features, structures, or characteristics of one or more other embodiments without limitation.

What is claimed is:

1. A method for determining blood vessel characteristic change using an ultrasonic sensor, the method comprising:
performing a plurality of ultrasonic signal transmit and receive operations at a position overlying a blood vessel of a person using an ultrasonic sensor, wherein the plurality of ultrasonic signal transmit and receive operations generate a plurality of received signals;
determining depths of blood vessel walls at the position for a plurality of time instances based on local maxima of a combination of an acoustic impedance mismatch and a motion characteristic based at least in part on the plurality of received signals; and
determining a change in a blood vessel characteristic based at least in part on a difference between the depths of the blood vessel walls at the plurality of time instances.

2. The method of claim 1, wherein the motion characteristic is a velocity of tissue, wherein the determining depths of blood vessel walls at the position for a plurality of time instances based on local maxima of a combination of an acoustic impedance mismatch and a motion characteristic based at least in part on the plurality of received signals comprises:
determining the velocity of the tissue at the plurality of time instances based at least in part on the plurality of received signals using at least a phase of the received signals.

3. The method of claim 2, wherein the determining a velocity of the tissue at the plurality of time instances based at least in part on the plurality of received signals using at least a phase of the received signals comprises:
performing Doppler signal processing on the plurality of received signals to determine the velocity of the tissue at the plurality of time instances.

4. The method of claim 2, wherein the determining depths of blood vessel walls at the position for a plurality of time instances based on local maxima of a combination of an acoustic impedance mismatch and a motion characteristic based at least in part on the plurality of received signals comprises:
determining a weighted velocity of the tissue at the plurality of time instances based on signal amplitudes of the plurality of received signals at the plurality of time instances and the velocity of the tissue at the plurality of time instances.

5. The method of claim 4, wherein the weighted velocity of the tissue depends on an impact of the acoustic impedance mismatch on the velocity of the tissue.

6. The method of claim 4, wherein the determining depths of blood vessel walls at the position for a plurality of time instances based on local maxima of a combination of an acoustic impedance mismatch and a motion characteristic based at least in part on the plurality of received signals comprises:
detecting two local maxima of the combination of the acoustic impedance mismatch and the motion characteristic based at least in part on the plurality of received signals, wherein the two local maxima correspond to the blood vessel walls.

7. The method of claim 6, wherein the detecting two local maxima of the combination of the acoustic impedance mismatch and the motion characteristic based at least in part on the plurality of received signals, wherein the two local maxima correspond to the blood vessel walls, comprises:
determining two depth ranges for the blood vessel based on blood vessel geometry, wherein a first depth range comprises a closer wall of the blood vessel relative to the ultrasonic sensor and a second depth range comprises a farther wall of the blood vessel relative to the ultrasonic sensor;
determining a first local maximum weighted velocity within the first depth range, wherein the first local maximum weighted velocity within the first depth range corresponds to the depth of the closer wall of the blood vessel; and
determining a second local maximum weighted velocity within the second depth range, wherein the second local maximum weighted velocity within the second depth range corresponds to the depth of the farther wall of the blood vessel.

8. The method of claim 7, wherein the blood vessel characteristic is a change in blood vessel diameter.

9. The method of claim 8, wherein the determining depths of blood vessel walls at the position for a plurality of time instances based on local maxima of a combination of an acoustic impedance mismatch and a motion characteristic based at least in part on the plurality of received signals further comprises:
determining a velocity of the blood vessel at the depth of the closer wall and a velocity of the blood vessel at the depth of the farther wall at the plurality of time instances, wherein the velocity of the blood vessel at the depth of the closer wall and the velocity of the blood vessel at the depth of the farther wall are out of phase; and
integrating the velocity of the blood vessel at the depth of the closer wall and the velocity of the blood vessel at the depth of the farther wall to generate a displacement of the closer wall and a displacement of the farther wall.

10. The method of claim 9, wherein the determining a change in a blood vessel characteristic based at least in part on a difference between the depths of the blood vessel walls at the plurality of time instances comprises:
calculating a diameter change of the blood vessel at the plurality of time instances based on a difference of the displacement of the closer wall and the displacement of the farther wall.

11. The method of claim 1, further comprising:
determining the motion characteristic at tissue at a depth between the ultrasonic sensor and a closer blood vessel wall relative to the ultrasonic sensor.

12. The method of claim 11, further comprising:
correcting for motion artifacts within displacement of the blood vessel walls by using the motion characteristic at tissue at a depth between the ultrasonic sensor and a closer blood vessel wall relative to the ultrasonic sensor.

13. The method of claim 1, wherein the blood vessel characteristic is a blood pressure.

14. The method of claim 1, wherein the blood vessel characteristic is a pulse wave velocity of the blood vessel.

15. An electronic device comprising:
an ultrasonic sensor
a memory; and
a processor configured to:
perform a plurality of ultrasonic signal transmit and receive operations at a position overlying a blood vessel of a person using the ultrasonic sensor, wherein the plurality of ultrasonic signal transmit and receive operations generate a plurality of received signals;
determine depths of blood vessel walls at the position for a plurality of time instances based on local maxima of a combination of an acoustic impedance mismatch and a motion characteristic based at least in part on the plurality of received signals; and
determine a change in a blood vessel characteristic based at least in part on a difference between the depths of the blood vessel walls at the plurality of time instances.

16. The electronic device of claim 15, wherein the processor is further configured to:
determine the velocity of the tissue at the plurality of time instances based at least in part on the plurality of received signals using at least a phase of the received signals.

17. The electronic device of claim 16, wherein the processor is further configured to:
determine a weighted velocity of the tissue at the plurality of time instances based on signal amplitudes of the plurality of received signals at the plurality of time instances and the velocity of the tissue at the plurality of time instances.

18. The electronic device of claim 17, wherein the processor is further configured to:
determine two depth ranges for the blood vessel based on blood vessel geometry, wherein a first depth range comprises a closer wall of the blood vessel relative to the ultrasonic sensor and a second depth range comprises a farther wall of the blood vessel relative to the ultrasonic sensor;
determine a first local maximum weighted velocity within the first depth range, wherein the first local maximum weighted velocity within the first depth range corresponds to the depth of the closer wall of the blood vessel; and
determine a second local maximum weighted velocity within the second depth range, wherein the second local maximum weighted velocity within the second depth range corresponds to the depth of the farther wall of the blood vessel.

19. The electronic device of claim 18, wherein the processor is further configured to:
determine a velocity of the blood vessel at the depth of the closer wall and a velocity of the blood vessel at the depth of the farther wall at the plurality of time instances, wherein the velocity of the blood vessel at the depth of the closer wall and the velocity of the blood vessel at the depth of the farther wall are out of phase; and
integrate the velocity of the blood vessel at the depth of the closer wall and the velocity of the blood vessel at the depth of the farther wall to generate a displacement of the closer wall and a displacement of the farther wall.

20. A non-transitory computer readable storage medium having computer readable program code stored thereon for causing a computer system to perform a method for determining blood vessel characteristic change using an ultrasonic sensor, the method comprising:
performing a plurality of ultrasonic signal transmit and receive operations at a position overlying a blood vessel of a person using an ultrasonic sensor, wherein the plurality of ultrasonic signal transmit and receive operations generate a plurality of received signals;
determining depths of blood vessel walls at the position for a plurality of time instances based on local maxima of a combination of an acoustic impedance mismatch and a motion characteristic based at least in part on the plurality of received signals; and
determining a change in a blood vessel characteristic based at least in part on a difference between the depths of the blood vessel walls at the plurality of time instances.
